(19) United States
(12) Patent Application Publication
(10) Pub. No.: US 2012/0148542 A1
(43) Pub. Date: Jun. 14, 2012

(54) MACHINE PERFUSION WITH COMPLEMENT INHIBITORS

(75) Inventor: David KRAVITZ, Barrington Hills, IL (US)

(73) Assignee: LIFELINE SCIENTIFIC, INC., Itasca, IL (US)

(21) Appl. No.: 13/315,556

(22) Filed: Dec. 9, 2011

Related U.S. Application Data
(60) Provisional application No. 61/421,948, filed on Dec. 10, 2010.

(51) Int. Cl. A01N 1/02; A61K 35/12

(52) U.S. Cl. 424/93.7; 435/1.2

(57) ABSTRACT

Methods for perfusing one or more organs, tissues or the like (hereinafter “organs”) with a composition comprising at least one complement inhibitor to sustain, maintain or improve the viability of the organs before and/or during transplantation.

### CROSS-REFERENCE TO RELATED APPLICATION

This nonprovisional application claims the benefit of U.S. Provisional Application No. 61/421,948 filed Dec. 10, 2010. The disclosure of the prior application is hereby incorporated by reference in its entirety.

### TECHNICAL FIELD

This disclosure particularly relates to processes and compositions for organ and tissue preservation and may include methods for perfusing one or more organs, tissues or the like (hereinafter “organs”) with a composition comprising at least one complement inhibitor to sustain, maintain or improve the viability of the organs before and/or during transplantation.

### BACKGROUND

Hypothermia is the bedrock of all useful methods of organ and tissue preservation, and has proven to be most effectively applied by controlling the extracellular environment of cells directly, and the intracellular environment indirectly, during cold exposure. Conventional control of the extracellular environment of cells to optimize preservation is based upon different strategies that include either static cold storage (or flush preservation), or low temperature continuous perfusion. These different strategies call for different approaches to interventional control of the extracellular environment in order to optimize preservation, and hence different design elements for the solutions used to effect these strategies.

Conventional cold flush storage or preservation is based upon the premise that temperature reduction to near but not below the ice point (0° C.) precludes the need to support metabolism to any significant extent, and that the correct distribution of water and ions between the intracellular and extracellular compartments can be maintained by physical rather than metabolic means. Thus, conventional solutions merely attempt to prevent or restrict cellular changes by manipulating the extracellular environment to abolish chemical potential gradients. On this basis, a variety of flush, or organ washout, solutions have been devised and evaluated for cold storage. These solutions are often referred to as “intracellular” solutions due to their resemblance, in some respects, to intracellular fluid.

With due consideration for the effects of ischemia, reperfusion, hypoxia, and hypothermia injury on cells, coupled with the proven efficacy of various existing organ preservation solutions, a general consensus of the most important characteristics in the design of hypothermic storage solutions has emerged. These include: minimizing of hypothermically induced cell swelling; preventing expansion of the interstitial space (especially important during perfusion); restricting ionic imbalances; preventing intracellular acidosis; preventing injury from free radicals; and providing substrates for regeneration of high energy phosphate compounds during reperfusion.

Attention to biophysical properties of “intracellular” flush solutions, to restrict passive diffusional processes, has unquestionably led to the development of techniques that have provided the basis of clinical organ preservation during the past few decades. However, improvements to the solutions used during organ preservation are still desired. The present disclosure describes further improvements of perfusion preservation methods and solutions that may be achieved by inclusion of complement inhibitors, which may be effective in attenuating, avoiding, and/or counteracting various destructive pathways, such as destructive pathways brought about by the deleterious effects of ischemia and reperfusion injury.

Thus, the methods of the present disclosure further improve conventional techniques for organ preservation by disclosing methods comprising perfusion of solutions including complement inhibitors, and optionally other biochemical and pharmacological components, and maintaining preservation in the dynamic state. Such complement inhibitors may be incorporated in the design of various solutions and/or perfusion solutions, such as the University of Wisconsin organ preservation solution (UW solution marketed as “Viaspan™”; DuPont), which has become the most widely used solution for cold flush preservation of kidneys, livers and pancreases.

Various complement inhibitors are known and may be incorporated into the methods of the present disclosure. One such complement inhibitor is APT070, or Microcept as described by Patel et al. J Am Soc Nephrol 17: 1102-1111, 2006. Other complement inhibitors that may be used in the present disclosure include those known in the art.

Patel et al., reported a strategy aimed to protect rat kidneys from complement-mediated postischemic damage and therefore increase the number of successful transplants. J Am Soc Nephrol 17: 1102-1111, 2006, which is hereby incorporated by reference in its entirety. Petal reported that rat donor kidneys were infused with a membrane-localizing complement regulator derived from human complement receptor type 1 (APT070) and then subjected to prolonged periods of static cold storage (at 4° C.). A relationship was found between the duration of cold ischemia and the extent of complement-mediated tubule damage and loss of graft function. After 16 h of cold static storage, APT070-treated kidneys that were transplanted into syngeneic recipients showed a significant increase in the number of surviving grafts, compared with control-treated grafts (63.6 versus 263%). Surviving grafts also displayed less acute tubular injury and better preservation of renal function. Unlike the methods of the present disclosure, Patel did not employ continuous perfusion preservation (such as continuous hypothermic perfusion preservation). Additionally, unlike the methods of the present disclosure, Patel did not add a complement inhibitor to any preservation perfusate while maintaining preservation in the dynamic state (e.g. machine perfusion).

In continuous perfusion preservation, the desirable properties of preservation solutions, such as hypothermic solutions listed above, are also applicable to controlling the extracellular environment by way of continuous perfusion techniques. In contrast to static cold storage, conventional continuous perfusion is usually controlled at around 10° C. and is based upon a different principle: it is generally assumed that a moderate degree of cooling will reduce metabolic needs but that continuous perfusion is required to support the suppressed metabolism and remove catabolic products (which may damage the cells). Because it is assumed that sufficient metabolic activity remains to actively regulate a near-normal cell volume and ionic gradients, the perfusates are generally acellular, isotonic, well oxygenated solutions having a composition that more closely resembles plasma than intracellular fluid. Such perfusates are therefore designated as “extracellular” solutions, and are perfused through the vascular bed of an organ at a pressure sufficient to achieve uniform tissue distribution (typically 40-60 mm Hg). To improve the effectiveness of conventional organ preservation methods, the present disclosure describes methods that may employ perfusion, such as substantially continuous perfusion during the preservation interval, of a least one perfusion solution that comprising at least one complement inhibitor to such perfusates, optionally with other additives and/or components, such as oncotic agents including, for example, albumin or synthetic macromolecular colloids, which may normally be incorporated into perfusates.

In addition to the principal objective of supporting metabolism, continuous perfusion also provides other advantages over flush and/or static preservation. These include the wash out of accumulated lactate and protons, thereby removing the metabolic block on glycolysis and potential components that may bring about further cell damage; this is thought to be especially beneficial for organs that have suffered prior warm ischemia. Perfusion also facilitates the removal of erythrocytes from the microcirculation and helps to maintain vascular patency during prolonged storage. Continuous perfusion has been shown to provide the best means of achieving prolonged hypothermic preservation (e.g., 3-7 days for kidneys), but concerns for damage to the vascular endothelium during prolonged perfusion may be a limiting factor.

Ideally organs are harvested in a manner that limits their warm ischemia time. Unfortunately, many organs, especially from non-beating heart donors, are harvested after extended warm ischemia time periods, e.g., 45 minutes or more. Machine perfusion of these organs at low temperature is preferable (Transpl Int 1996 Daemen). Further, low temperature machine perfusion of organs at low pressures is also preferable (Transpl. Int 1996 Yland). Roller or diaphragm pumps are often used to deliver perfusate at controlled pressures. Numerous control circuits and pumping configurations are used to achieve preferable perfusion conditions. See, for example, U.S. Pat. Nos. 5,338,662 and 5,494,822 to Sadri; U.S. Pat. No. 4,745,759 to Bauer et al.; U.S. Pat. Nos. 5,217,860 and 5,472,876 to Fahy et al.; U.S. Pat. No. 5,051,352 to Martindale et al.; U.S. Pat. No. 3,995,444 to Clark et al.; U.S. Pat. No. 4,629,686 to Gruenberg; U.S. Pat. Nos. 3,738,914 and 3,892,628 to Thorne et al.; U.S. Pat. Nos. 5,285,657 and 5,476,763 to Bacchi et al.; U.S. Pat. No. 5,157,930 to McGhee et al.; and U.S. Pat. No. 5,141,847 to Sugimachi et al.

During organ perfusion preservation, a goal is to perfuse fluids through the vessels of the ex vivo organ (1) in sufficient volume, i.e., flow, to enable proper dilution of waste products and proper provision of nutrients; and (2) at sufficient pressure to maintain vessel patency while limiting maximum flow and pressure to avoid damage. The present disclosure includes at least one complement inhibitor in one or more of the perfusate solutions that a donor organ is exposed to (or contacted with) in order to improve upon the conventional perfusion preservation methods and to help avoid damage to the donor organ.

### SUMMARY

A need exists for improved methods of preserving an organ by avoiding damage to the organ and maintaining the organ's viability.

This disclosure is directed to methods for preserving an organ by perfusing one or more organs, tissues or the like (hereinafter “organs”) with a composition and/or solution comprising at least one complement inhibitor to sustain, maintain or improve the viability of the organs before and/or during transplantation.

In embodiments, the solution comprising at least one complement inhibitor may be a base perfusate solution, such as, for example, a hypothermic physiologic solution, or mid- or normothermic oxygen carrying or non-oxygen carrying solution. In embodiments, a solution comprising at least one compound and/or complement inhibitor is perfused so as to deliver a homogeneous distribution of the at least one compound and/or complement inhibitor throughout the organ or tissue, where an effective amount of at least one complement inhibitor is contained in the solution to have the desired effect being that upon transplant, said organ or tissue, having been administered the at least one compound and/or complement inhibitor, is less likely to experience delayed graft function, deleterious effects of ischemia/reperfusion injury, including inflamatory reactions, and/or other detrimental responses that can injure the organ or recipient including precipitating or enhancing an immunological reaction from the recipient with the potential of compromising the graft's and/or recipient's short term and/or long term health and proper functionality.

In embodiments, the methods of the present disclosure comprise steps in which the donor organ is evaluated while being treated on the perfusion apparatus for specific biological markers of progress (at various ex vivo pump time intervals). In embodiments, the methods of the present disclosure comprise steps in which a distribution of an added compound, such as a complement inhibitor, is assessed and its presence throughout the entire organ is quantified. In embodiments, the methods of the present disclosure comprise steps in which the organ's acceptance of the compound, such as a complement inhibitor, or effect thereof, at cellular or sub cellular levels is assessed, monitored and/or quantified.

These and other features and advantages of the disclosed methods and systems are described in, or apparent from, the following detailed description of various exemplary embodiments.

### DETAILED DESCRIPTION OF PREFERRED EMBODIMENTS

The basic principle of cellular preservation for clinical application is to minimize the deleterious effects that cells may experience. This can either be achieved pharmacologically by using a wide variety of cytoprotective drugs, and/or by reducing temperature. Conventional wisdom teaches us that there is no single drug, or cocktail of drugs, that can so safely and effectively suppress metabolism and provide ischemic protection for multiple tissues and organs as the application of hypothermia can. Accordingly, the focus changes to control the respective environment of the cells to optimize preservation. The methods of the present disclosure improve upon the conventional methods of organ preservation by use of improved methodology in which at least one compound and/or complement inhibitor is added to a base perfusion solution that is perfused through the donor organ.

As used herein, the term “perfusion” means the flowing of a fluid through the organ or tissue. Techniques for perfusing organs and tissues are discussed in, for example, U.S. Pat. No. 5,723,282 to Fahy et al., which is incorporated herein in its entirety.

In embodiments, the methods disclosed herein implement a new approach that utilizes advances in perfusion technology and combines those advances with solutions comprising complement inhibitors, such as blood substitute solutions comprising at least one complement inhibitor to improve the preservation process. This approach circumvents several recognized shortcomings in the present modes of clinical organ storage, the most notable of which are the shortcomings of the conventional two layer method (TLM).

Various complement inhibitors are known and may be incorporated into the methods of the present disclosure. One such complement inhibitor is APT070, or Microcept as described by Patel et al. J Am Soc Nephrol 17: 1102-1111, 2006. Other complement inhibitors that may be used in the methods of the present disclosure include those known in the art, such as the compounds and/or complement inhibitors described in U.S. Pat. Nos. 5,562,904; 5,624,837; 5,627,264; 5,643,770; 5,719,127; 5,843,884; 5,847,082; 6,166,288; 6,248,365; 6,270,997; 6,399,105; 6,458,360; 6,479,652; 6,503,947; 6,825,395; 7,485,769; 7,534,448; 7,560,279; 7,674,769; 7,807,175; and 7,829,659, and U.S. Patent Application Publication Nos. 2006/0178308 and 2007/0141573, the entire disclosures of which are hereby incorporated by reference in their entireties. In embodiments, hydroxilase inhibitors may also be added to the solutions of the present disclosure.

Methodology for assessing and/or quantifying and/or measuring complement coverage is known. Suitable methodology for assessing and/or quantifying and/or measuring complement coverage in accordance with the methods of the present disclosure may be found within the above-identified references, and include analysis methods, such as, for example, staining; extraction; various absorbance, fluorescence, phosphorescence, and transmission spectroscopic methods, spectrophotometric methods, and spectrometric methods; mass spectrometry; and the like, and/or combinations thereof.

In embodiments, organs may be exposed to or contacted with solutions comprising at least one complement inhibitor during machine perfusion, such as Hypothermic Machine Perfusion (HMP). Conventional methods of organ preservation for transplantation rely principally upon static cold storage on ice, a relatively simple and economic technique that has been used for several decades. However, modern day demands for increasing the numbers of organs available for transplant has led to a resurgence of interest in hypothermic perfusion preservation (HPP) of organs because perfusion techniques provide significant advantages over static cold storage. In this context HPP is based upon the fundamental premise that devices can be designed to facilitate the replacement of blood in the circulation of an, ex vivo organ with specially designed fluids to maximize the protective effects of hypothermia on the ischemic tissue. This approach has the potential, and has already been shown in many applications, to circumvent some of the recognized shortcomings of conventional cold storage.

The methods of the present disclosure are involved in transport, storage, perfusion and diagnosis of organs. However, the disclosed methods may have other applications, and thus should not be construed to be limited to particular contexts of use. Various disclosed features may be particularly suitable for use in the context of, and in conjunction and/or connection with the features of the apparatus and methods disclosed in U.S. patent application Ser. Nos. 09/162,128 (now abandoned); 12/926,277; and 12/910,308, U.S. Pat. Nos. 6,977,140 and 6,673,594, and U.S. Patent Application Publications Nos. 2004-0248281, 2004-0221719, and 2004-0111104, the disclosures of which are hereby incorporated by reference herein in their entirety.

Various perfusion apparatus, which may be used in the methods of the present disclosure, for organs are described in the scientific and patent literature. For example, U.S. Pat. No. 6,977,140, assigned to Organ Recovery Systems, Inc., which is hereby incorporated by reference herein in its entirety, describes such an apparatus. An exemplary apparatus that may be used in the present disclosure is also described in U.S. patent application Ser. No. 12/379,239, which is a division of U.S. patent application Ser. No. 11/075,690, filed Mar. 10, 2005 (issued as U.S. Pat. No. 7,504,201 on Mar. 17, 2009) the entire disclosures of which are hereby incorporated by reference in their entireties. In embodiments, the methods described herein employ the LifePort® platform transporter or a modified LifePort® platform transporter in order to accomplish hypothermic machine perfusion (HMP) of a donor tissue or organ.

Briefly, in an organ perfusion apparatus, gross organ perfusion pressure may be provided by a pneumatically pressurized medical fluid reservoir controlled by a computer. The computer may be programmed to respond to an input from a sensor or similar device, for example, disposed in a flow path such as in an end of tubing placed in a vessel of the perfused organ. The computer may be used in combination with a stepping motor/cam valve or pinch valve to (1) enable perfusion pressure fine tuning, (2) prevent overpressurization, and/or (3) provide emergency flow cut-off in the vessel. Alternatively, the organ may be perfused directly from a computer controlled pump, such as a roller pump or a peristaltic pump, with proper pump control and/or sufficient fail-safe controllers to prevent overpressurization of the organ, especially as a result of a system malfunction. Substantially eliminating overpressurization potential may reduce the consequent potential damage to the vascular endothelial lining, and to the organ tissue in general, and mitigate the effects of flow competition and flow extinction in a lower pressure vessel.

An organ diagnostic apparatus may also be provided to produce diagnostic data such as an organ viability index and/or compound and/or complement coverage. The organ diagnostic apparatus may include features of an organ perfusion apparatus, such as sensors and temperature controllers, as well as cassette interface features. The organ diagnostic apparatus may provide analysis of input and output fluids in a perfusion system. Typically, the organ diagnostic apparatus is a simplified perfusion apparatus providing diagnostic data in a single pass, in-line perfusion.

Disclosed embodiments may also provide an organ cassette that allows an organ to be easily and safely moved between apparatus for perfusing, storing, analyzing and/or transporting the organ. The organ cassette may be configured to provide uninterrupted sterile conditions and efficient heat transfer during transport, recovery, analysis and storage, including transition between the transporter, perfusion apparatus and/or organ diagnostic apparatus, and/or other apparatus.

Disclosed embodiments may also provide an organ transporter that allows for transportation of an organ, particularly over long distances. The organ transporter may include features of an organ perfusion apparatus, such as sensors and temperature controllers, as well as organ cassette interface features.

Disclosed embodiments of the perfusion apparatus, transporter, cassette, and organ diagnostic apparatus may be networked to permit remote management, tracking and monitoring of the location and therapeutic and diagnostic parameters of the organ being stored or transported. Information systems may be used to compile historical data of organ transport and storage, including amounts and types of complement inhibitors to which the donor organ has been exposed, and provide cross-referencing with hospital and United Network for Organ Sharing (UNOS) data on the donor and recipient for the organ. The information systems may also provide outcome data to allow for ready research of perfusion parameters and transplant outcomes. Such outcome data may correlate the amounts and types of complement inhibitors to which the donor organ has been exposed to transplant outcomes.

The perfusion apparatus may be at least partially microprocessor controlled, and pneumatically actuated. A microprocessor with connections to sensors, valves, thermoelectric units and pumps may be used in the methods of the present disclosure. The microprocessor and apparatus may be configured to be further connected to a computer network to provide data sharing, for example, across a local area network or across the Internet.

In embodiments, the apparatus may be capable of perfusing one or more organs simultaneously, at both normothermic and hypothermic temperatures. All solution or medical fluid contact surfaces may be formed of, or coated with, materials compatible with the medical fluid used, more preferably non-thrombogenic materials. The apparatus may include a housing that includes front cover, which may be translucent, and a reservoir access door. The apparatus may include one or more control and display areas for monitoring and controlling perfusion.

In embodiments, enclosed within the housing is a reservoir that may include multiple reservoir tanks such as three or more reservoir tanks. The reservoir tanks may be standard one liter infusion bags, each with a respective pressure cuff. A pressure source may be provided for pressurizing the pressure cuffs. The pressure source may be pneumatic and may include an onboard compressor unit supplying external cuff activation via gas tubes. Disclosed embodiments, however, are not limited to use of an onboard compressor unit as any adequate pressure source can be employed. Other available pressures sources may include a compressed gas (e.g., air, CO₂, oxygen, nitrogen, etc.) tank. Alternatively, an internally-pressurized reservoir tank may be used. In embodiments, the reservoir tanks may be bottles or other suitably rigid reservoirs that can supply perfusate by gravity or can be pressurized by compressed gas.

Gas valves may be provided on respective gas tubes to allow for control of the pressure provided by the onboard compressor unit. Anti-backflow valves may also be provided respectively on gas tubes. Pressure sensors may be provided to relay detected pressure conditions to the microprocessor. Corresponding flow sensors may also be provided. The perfusion, diagnostic and/or transporter apparatus may be provided with sensors to monitor perfusion fluid pressure and flow in the particular apparatus to detect faults in the particular apparatus, such as pressure elevated above a suitable level for maintenance of the organ. Gas valves may be provided to release pressure from the cuffs. One or more gas valves may be vented to the atmosphere. A gas valve may also be in communication with reservoir tanks via tubing, which may be provided to vent air from the reservoir tanks. Various tubing may be configured with filters and/or check valves to prevent biological materials from entering the tubing or from proceeding along the fluid path. The check valves and/or filters may be used to prevent biological materials from leaving one organ perfusion tubeset and being transferred to the tubeset of a subsequent organ in a multiple organ perfusion configuration. The check valves and/or filters may also be used to prevent biological materials, such as bacteria and viruses, from being transferred from organ to organ in subsequent uses of the perfusion apparatus in the event that such biological materials remain in the perfusion apparatus after use. The check valves and/or filters may be provided to prevent contamination problems associated with reflux in the gas and/or vent lines. For example, the valves may be configured as anti-reflux valves to prevent reflux. An additional third reservoir tank may be pressurized by pressure released from one of the pressure cuffs via further gas valve.

The solution or medical fluid that may comprise the at least one complement inhibitor may be a natural fluid, such as blood, or otherwise synthetic fluid, which may, for example, be a simple crystalloid solution, or may be augmented with an appropriate oxygen carrier. In embodiments, the solution or medical fluid that may comprise the at least one complement inhibitor may be prepared by mixing a first portion of the solution, which may be a medical fluid and/or base perfusion solution, with at least one complement inhibitor to produce a first mixed solution. In embodiments, the first mixed solution is perfused through an organ or tissue. In embodiments, a second mixed solution, third mixed solution, fourth mixed solution, fifth mixed solution, etc., which may be prepared in a similar manner as the first mixed solution, and may also be perfused through an organ or tissue. In embodiments, one or more of the second mixed solution, third mixed solution, fourth mixed solution, fifth mixed solutions may be identical to the first mixed solution. In embodiments, one or more of the second mixed solution, third mixed solution, fourth mixed solution, fifth mixed solutions may be different from the first mixed solution and either comprise different concentrations of the respective components comprised in the first mixed solution and/or comprise more or fewer components than the first mixed solution. In embodiments, one or more solutions and/or medical fluids that do not comprise a complement inhibitor may also be perfused through an organ or tissue either before or after any of the above described mixed solutions. In embodiments, increasing the O₂ content any of the solutions, replenishing the O₂ content any of the solutions, and/or introducing an O₂ content into any of the solutions described in the present disclosure may occur by methods known of to those of ordinary skill in the art, such as by use of a known oxygenator.

In embodiments, an oxygen supply may be coupled to an oxygenator through a pressure regulator. The oxygenator may be attached to the side of a reservoir or a container comprising the organ. Similarly, the bubble remover may be attached to a container comprising the organ. The bubble remover may also be independent of a container comprising the organ or integrated into a container comprising the organ or another part of the apparatus.

In embodiments, the oxygen carrier may, for example, be washed, stabilized red blood cells, cross-linked hemoglobin, pegolated hemoglobin or fluorocarbon based emulsions. In embodiments, the solution or medical fluid that may comprise the at least one complement inhibitor may also contain antioxidants known to reduce peroxidation or free radical damage in the physiological environment and specific agents known to aid in organ protection. In embodiments, an oxygenated, e.g., cross-linked hemoglobin-based bicarbonate, solution that may comprise the at least one complement inhibitor may be used for a normothermic mode while a non-oxygenated, e.g., simple crystalloid solution, such as one augmented with antioxidants, solution that may comprise the at least one complement inhibitor may be used for a hypothermic mode. In embodiments, the solutions or medical fluids (that may comprise the at least one complement inhibitor) used in both the normothermic and hypothermic modes may be designed or selected to reduce or otherwise prevent the washing away of, or damage to, the vascular endothelial lining of the organ. For a hypothermic perfusion mode, as well as for flush and/or static storage, an exemplary solution to which at least one complement inhibitor may be added is disclosed in U.S. Pat. No. 6,492,103, the disclosure of which is hereby incorporated herein by reference in its entirety. Examples of additives which may be used in perfusion solutions, which may comprise at least one complement inhibitor, are also disclosed in U.S. Pat. No. 6,046,046 to Hassanein, the disclosure of which is hereby incorporated by reference herein in its entirety. Other suitable solutions and materials may also be used.

In embodiments, the methods of the present disclosure may be accomplished by incorporating one or more complement inhibitors into known systems and solutions, such as KPS-1™ and SPS-1™ available from Organ Recovery Systems, and/or those taught in the Brasile and Taylor patents (U.S. Pat. Nos. 5,643,712; 5,699,793; 5,843,024 to Brasile and U.S. Pat. Nos. 5,599,659; 5,702,881 to Brasile et al.; U.S. Pat. Nos. 5,405,742 and 5,514,536 to Taylor, each of which is incorporated herein by reference in its entirety), which have required completely different compositions for different stages of organ procurement, preservation and transplantation procedures. Alternatively, the methods of the present disclosure may be accomplished by incorporating one or more complement inhibitors into one or two (or optionally more) base compositions (such as the base compositions disclosed in U.S. Pat. Nos. 6,994,954 and 6,492,103 to Taylor each of which is incorporated herein by reference in its entirety) and a number of different additives that can be added to one or more of the base compositions to produce specific compositions useful for specific stages in organ or tissue procurement, preservation and transplantation. In embodiments, the base solution and additives, including, for example, complement inhibitors, may be stored in separate containers in a single package or kit. In embodiments, the at least one complement inhibitor may be present in the above solutions and/or compositions in a sufficient amount to assure complete complement coverage and substantially eliminate cellular damage.

A base formulation, which may comprise at least one complement inhibitor, may include a design that takes into account the biophysical and minimal biochemical components that can be standardized for all or a desired subset of applications. Such a base solution may then be used as a vehicle for a range of additive “cocktails,” including complement inhibitors, to derive a system of solutions, each of which may be used at various stages during organ preservation, transport and transplantation, optimized for different needs. In embodiments, the at least one complement inhibitor may be present in the base solutions in a sufficient amount to assure complete complement coverage and substantially eliminate cellular damage.

In embodiments, solutions for warm ischemic time-organ preservation may include adding at least one complement inhibitor (which may or may not be the same complement inhibitor) to, for example, one or more of the following solutions: a hypothermic flush/purging solution, a hypothermic perfusate/maintenance solution, a “Normothermic” perfusate/rescue solution and/or a pre-reimplantation flush/rinse solution. In embodiments, one or more of the above solutions, which may include at least one complement inhibitor (which may or may not be the same complement inhibitor) may be perfused through the donor organ before and/or during transplantation procedures.

Exemplary embodiments may include, for example, adding the at least one complement inhibitor to the following base solutions:

Intracellular Base Solution: minimum requirements for cold storage including cryopreservation solutions. An exemplary formulation of such a solution is given in Table 2 (below).

Extracellular Base Solution: plasma-like electrolytes as base for oxygen carrying molecules and other substrates necessary for optimized “normothermic” perfusion.

Exemplary embodiments may include, for example, adding at least one complement inhibitor to the following base plus additive solutions:

Purge=Extracellular Base plus purge additive (“cocktail”) designed principally to purge the vasculature of blood in preparation for preservation.

Maintenance=Intracellular Base plus cytoprotection additive (“cocktail”) designed to protect and maintain cellular stability during cold storage. Ideally, this will apply to both static cold storage and cold machine perfusion.

Rescue=Extracellular Base plus rescue additive (“cocktail”) for near normothermic perfusion.

Rinse=Extracellular Base plus rinse additive (“cocktail”) designed to flush out unwanted preservation molecules prior to reimplantation. This may fulfill a different role from the Purge solution designed to remove erythrocytes and other blood components prior to the preservation phase.

Cryo=Concentrated Intracellular Base plus permeating or non-permeating cryoprotective additives for sub-zero preservation of cells and tissues. For the Cryo embodiment, the intracellular base is preferably concentrated to a 3× to 4× strength in comparison to its use alone and with most other additives. This facilitates its combination with additive cryoprotective compounds.

In embodiments, the at least one complement inhibitor may be present in the solution and/or composition being perfused in an effective and/or sufficient amount to assure complete complement coverage of the organ or tissue. In embodiments, the at least one complement inhibitor may be present in the solution and/or composition being perfused in an effective and/or sufficient amount to (1) assure complete complement coverage of the organ or tissue, and (2) substantially prevent and/or completely prevent cellular damage. In embodiments, the at least one complement inhibitor is administered by perfusion techniques in an effective and/or sufficient amount to assure complete complement coverage of the organ or tissue. In embodiments, the at least one complement inhibitor is administered by perfusion techniques in an effective and/or sufficient amount to (1) assure complete complement coverage of the organ or tissue, and (2) substantially prevent and/or completely prevent cellular damage.

In embodiments, the methods of the present disclosure comprise perfusing an organ or tissue (which optionally may also be referred to hereinafter as a “donor organ”) with one or more of the solutions described in the present disclosure (which may comprise at least one complement inhibitor). As used herein “solution” encompasses, for example, any solution, such as a base perfusion solution, unless otherwise specifically limited. In embodiments, multiple solutions that are not identical are perfused into the donor organ, where at least one of the solutions that are perfused into the donor organ contains at least one complement inhibitor. In embodiments, multiple solutions that are not identical are perfused into the donor organ. In embodiments, each of the solutions that are perfused into the organ contains the same complement inhibitor. In embodiments, multiple solutions that are not identical are perfused into the donor organ and each of the different solutions that are perfused into the organ contains a complement inhibitor that is not identical to the complement inhibitor contained in the other solutions. In embodiments, at least one of the solutions that are perfused into the organ contain a second complement inhibitor and/or a third, fourth, fifth, etc., that is different from each other and/or the combination of complement inhibitors in any of the other solutions.

In embodiments, the at least one complement inhibitor is comprised in a solution that is introduced and/or perfused through the donor organ before any cooling of the donor organ occurs. In embodiments, the at least one complement inhibitor is comprised in a solution that is introduced and/or perfused through the donor organ before cooling the donor organ to a predetermined temperature, such as before the organ is cooled to a temperature less than about 15° C., or before the organ is cooled to a temperature less than about 10° C., or before the organ is cooled to a temperature less than about 5° C., or before the organ is cooled to a temperature less than about 0° C.

In embodiments, the at least one complement inhibitor is comprised in a solution that is only introduced and/or perfused through the donor organ while the organ is maintained a high and/or mid predetermined temperature range, such as from about 10° C. to about 37° C., or from about 15° C. to about 37° C., or from about 20° C. to about 37° C., or from about 20° C. to about 30° C., or from about 20° C. to about 25° C.

In embodiments, the at least one complement inhibitor is comprised in a solution that is only introduced and/or perfused through the donor organ while the organ is maintained at a low and/or mid predetermined temperature range, such as from about 0° C. to about 20° C., or from about 10° C. to about 20° C., or from about 10° C. to about 15° C., or from about 0° C. to about 15° C., or from about 0° C. to about 10° C., or from about 0° C. to about 5° C., or at a temperature less than about 0° C.

In embodiments, the at least one complement inhibitor is comprised in a solution that is introduced and/or perfused through the donor organ after cooling the donor organ, such as after the organ has been cooled to a temperature less than about 15° C., or after the organ has been cooled to a temperature less than about 10° C., or after the organ has been cooled to a temperature less than about 5° C., or after the organ has been cooled to a temperature less than about 0° C.

In embodiments, the donor organ is not exposed to the complement inhibitor until the organ has been cooled to a predetermined temperature, such as a temperature less than about 20° C., or a temperature less than about 10° C., or a temperature from about 0° C. to about 10° C., or a temperature less than about 0° C. In such embodiments, the at least one complement inhibitor is comprised in a solution that is initially introduced and/or perfused through the donor organ after cooling the donor organ by perfusing the donor organ with one or more preservation solutions that do not contain any complement inhibitors. For example, the at least one complement inhibitor may be included in a solution that is initially introduced and/or perfused through the donor organ after cooling the donor organ (such as by perfusing the donor organ with a different preservation solution that does not contain complement inhibitor) to a temperature of less than about 15° C., or after the organ has been cooled to a temperature less than about 10° C., or after the organ has been cooled to a temperature less than about 5° C., or after the organ has been cooled to a temperature less than about 0° C. In alternative embodiments, before the donor organ is cooled, the organ is perfused with a solution that may contain at least one complement inhibitor.

In embodiments, the at least one complement inhibitor is comprised in a solution that is introduced and/or perfused through the donor organ during a re-warming process, such as, for example, a re-warming process that occurs after the organ has been cooled, such as after the organ has been cooled to a predetermined temperature.

In embodiments, the at least one complement inhibitor is comprised in a solution that is initially introduced and/or perfused through the donor organ during a re-warming process that occurs after the organ has been cooled to a predetermined temperature. In specific embodiments, the donor organ is not exposed to the complement inhibitor until the donor organ experiences a re-warming process that occurs after the organ has been cooled to a predetermined temperature, such as a temperature less than about 20° C., or a temperature less than about 10° C., or a temperature from about 0° C. to about 10° C., or a temperature less than about 0° C. In embodiments, the donor organ may not be exposed to a complement inhibitor until a re-warming process occurs and the organ is perfused with a solution comprising at least one complement inhibitor during the re-warming process while the donor organ is being maintained a temperature from about 0° C. to about 37° C., such as a temperature from about 10° C. to about 37° C., or a temperature from about 20° C. to about 37° C.

In embodiments, the solution contains an effective amount of the at least one complement inhibitor to protect against tissue damage. In embodiments, the solution contains an effective amount of the at least one complement inhibitor to prevent or substantially prevent delayed graft function, deleterious effects of ischemia/reperfusion injury, including inflamatory reactions, and/or other detrimental responses that can injure the organ or recipient. In embodiments, the solution contains an effective amount of the at least one complement inhibitor to prevent or substantially prevent delayed graft function, deleterious effects of ischemial/reperfusion injury, including inflamatory reactions, and/or other detrimental responses that can injure the organ or recipient, which including precipitating or enhancing an immunological reaction from the recipient with the potential of compromising the graft's and/or recipient's short term and/or long term health and proper functionality. In embodiments, the at least one complement inhibitor represents from about 0.000001% to about 0.5% of the total weight of the solution (the term solution, as has used herein and throughout the present disclosure, may refer to a base perfusion solution), such as from about 0.00001% to about 0.1% of the total weight of the base perfusion solution, or from about 0.0005% to about 0.05% of the total weight of the base perfusion solution. In embodiments, the at least one complement inhibitor is present in the solution (which, as discussed above, may be a base perfusion solution) in an amount greater than about 0.01 μg/ml, such as in an amount greater than about 0.1 μg/ml, or in an amount greater than about 1 μg/ml, or in an amount greater than about 10 μg/ml, or in an amount greater than in an amount greater than about 200 μg/ml. In embodiments, the at least one complement inhibitor is present in the solution (which, as discussed above, may be a base perfusion solution) in an amount of from about 0.01 μg/ml to about 500 μg/ml, such as in an amount of from about 0.1 μg/ml to about 150 μg/ml, or in an amount of from about 1 μg/ml to about 100 μg/ml, or in an amount of from about 10 μg/ml to about 50 μg/ml. In embodiments, the above solutions comprising the at least one complement inhibitor may be perfused into an organ or tissue until a desired dosage (such as an effective dosage) of the at least one complement inhibitor is present in the organ or tissue to prevent and/or protect against tissue damage.

In embodiments, at least one complement inhibitor is administered by perfusion in an amount effective to prevent and/or protect against tissue damage. In embodiments, the at least one complement inhibitor is administered by perfusion in an amount effective to prevent or substantially prevent delayed graft function, deleterious effects of ischemia/reperfusion injury, including inflamatory reactions, and/or other detrimental responses that can injure the organ or recipient. In embodiments, the at least one complement inhibitor is administered by perfusion in an amount effective prevent or substantially prevent delayed graft function, deleterious effects of ischemia/reperfusion injury, including inflamatory reactions, and/or other detrimental responses that can injure the organ or recipient, which including precipitating or enhancing an immunological reaction from the recipient with the potential of compromising the graft's and/or recipient's short term and/or long term health and proper functionality.

In embodiments, the changes in the concentration of the at least one complement inhibitor present in the solution may be made during perfusion in any manner, such as by way of a stepwise change, or gradient change in which a respective component's concentration is gradually increased to the desired concentration, or decreased to the desired concentration, or decreased to the point of elimination from the solution being perfused, if necessary.

In embodiments, the methods disclosed herein utilize a combination of technologies in HMP and HBS along with the merits of PFC oxygenation to generate a new hybrid technique that solves the problems of static cold storage methods having a perfluorochemical layer. Selection of the baseline medium or perfusate in which to deliver the PFC as an emulsion also demands consideration of what will be optimal for the respective cell (e.g., pancreatic cells, cardiac cells, etc.) preservation under hypothermic conditions. To this end, this disclosure includes the preparation of preservation solutions including at least one complement inhibitor designed as hypothermic blood substitutes.

Hypothermic blood substitutes as preservation media: Traditionally, a variety of organ preservation solutions have been developed. U.S. Pat. Nos. 5,643,712, 5,699,793, 5,843,024 to Brasile and Nos. 5,599,659, 5,702,881 to Brasile et al., the disclosures of each of which are incorporated herein by reference in their entireties, describe separate resuscitation and preservation solutions for tissues and organs. The Brasile patents disclose compositions to which at least one complement inhibitor may be added, if desired, that may be used in methods of this disclosure.

Taylor et al. have formulated and evaluated two solutions designated Hypothermosol™-purge (HTS-P) and Hypothermosol™-maintenance (HTS-M). Some aspects of these solutions are described in U.S. Pat. Nos. 5,405,742 and 5,514,536 to Taylor, the disclosures of both of which are incorporated herein by reference in their entireties. The Taylor patents disclose compositions to which at least one complement inhibitor may be added, if desired, that may be used in methods of this disclosure.

The protective properties of solutions such as the Unisol® family of solutions (as described in U.S. Pat. Nos. 6,492,103 and 6,994,954, entitled “System for organ and tissue preservation and hypothermic blood substitution” to Taylor, the disclosures of which are hereby incorporated by reference in their entireties) may be used in methods of this disclosure. In embodiments, Unisol may be mixed with at least one complement inhibitor, if desired, and may be utilized as the vehicle solution for emulsifying PFCs to significantly increase its oxygen delivery capacity, in addition to cytoprotective additives.

In embodiments, the perfusion solution may be a hyperkalemic, “intracellular-type” solution designed to “maintain” cellular integrity during hypothermic exposure at the nadir temperature (<10° C.). In embodiments, such a solution may comprise at least one complement inhibitor.

Increasing oxygen delivery to tissues during hypothermic storage and the role of PFCs: The Unisol® “maintenance” solution was developed and tested at temperatures in the range of 7-10° C., which conforms with the temperature range in which ATP reserves can be re-established if an adequate supply of O₂ is maintained by continuous perfusion. For example, numerous investigations have suggested that oxygen supply is essential during hypothermic preservation of organs, such as livers.

The rapid depletion of adenine nucleotides during cold storage of organs at 0-2° C. (e.g. conventional static cold ice-storage) may be suggestive that mitochondrial function is severely impaired by hypothermia. These levels of O₂ may need to be sustained during perfusion to ensure the highest donor organs and the use of PFCs in the methods of the present disclosure may allow for this to be accomplished.

PFCs are hydrocarbons in which all or most of the hydrogen atoms are replaced with fluorine (e.g., perfluorocarbons). They have twice the density of water and a high capacity for dissolving respiratory gases. The solubility of dissolved oxygen in PFC is approximately 25 times greater than in blood or water. The ability of PFCs to release oxygen in accordance with Henry's Law is not significantly influenced by temperature, making them ideal for delivering oxygen during hypothermic organ preservation. This is also supported by recent demonstrations that the gas-dissolving and gas-unloading properties of perfluorocarbon were necessary in a peritoneal perfusion application for systemic oxygenation since the same effect was not obtained when saline solution alone was employed as the perfusate. However, the use of perfluorocarbon under hypothermic conditions has been limited.

Further improvements and benefits to the methods of the present may occur by optimizing the composition of these baseline perfusates by adding cytoprotective agents design to minimize preservation and reperfusion injury, and/or PFCs. For example, cytoprotective additives may be additives displaying efficacy during low temperature preservation and therefore a high probability they will have a positive impact on the quality of pancreas preservation during hypothermic machine perfusion, such as antioxidants, anti-apoptotic agents and trophic factors.

Antioxidants: Oxygen-derived free radicals (ODFR) have been the focus of attention as mediators of various tissue injuries and particularly microvascular injury. It is possible for the production of injurious free radicals to be enhanced during cold storage, it is important to appreciate that the resultant cell damage may not occur entirely at the low temperature. On the contrary there is a growing body of evidence that reintroduction of oxygenation via a regular blood supply upon re-warming and reperfusion provides a powerful impetus for further oxidative stress. A principal pathway is the stimulation of enzymically driven radical reactions such as the xanthine/xanthine oxidase system involving the interaction of ATP catabolic products with molecular oxygen. Vascular endothelial cells are thought to be particularly vulnerable to this type of injury mediated by free radical generation by this so-called “respiratory burst” mechanism. Nevertheless, low concentrations of molecular oxygen such as that dissolved in organ preservation solutions may be sufficient to support the generation of free radicals during prolonged storage. Therefore, without the proper balance of antioxidants, cold exposure may set the stage for a progressive development of tissue injury as a result of reactions and processes that occur during hypothemiia.

In embodiments, the antioxidants may be present in a sufficient amount to substantially eliminate cellular damage and/or oxidative stress.

Whilst cells employ a number of repair mechanisms to recover from injuries occurring as a result of free radical activity, cell survival depends upon whether salvage pathways are overwhelmed or whether a point of irreversible damage is reached during the storage/reactivation process such that cell death becomes inevitable. Accordingly, in embodiments, the antioxidants, and amounts thereof, are selected to circumvent oxidative stress and reperfusion injury under both hypothermic and normothermic conditions. Exemplary antioxidants may include dibutyryl-cAMP (db-cAMP), α-tocopherol (Vitamin E), Trolox™, and hypothermosol plus both EDTA and Vitamin E.

Anti-Apoptotic Agents: While many of the diverse stresses known to cause necrotic cell death have also been reported to induce apoptosis in a variety of cells, the role of low temperatures as a possible stimulus of programmed cell death has only recently begun to emerge. It is now established that apoptosis plays an integral role in cell death induced by the rigors of both hypothermia and cryopreservation. More specifically, apoptosis has been identified to be directly associated with delayed-onset cell death (DOCD). This is defined as death associated with cold exposure that is not apparent immediately upon re-warming, but extending over the post-exposure recovery period. Recent research into the causative apoptotic and necrotic pathways responsible for low temperature induced DOCD has identified the contribution of multiple apoptotic pathways, including receptor- and mitochondrial-induced apoptosis. Investigations into these pathways, their progression, and their induction stressors has begun to facilitate new methods for improving preservation efficacy through the modulation of the cellular and molecular responses of a cell undergoing preservation (both hypothermic and cryopreservation).

Incorporation of specific apoptotic protease inhibitors in preservation media has now been reported to markedly improve the survival of a variety of cells and tissues. Furthermore, investigation into the modification of the carrier medium from that of standard extracellular-type culture media with, or without cryoprotectants, to that of specifically designed intracellular-type preservation solutions such as Unisol™, or its predecessor Hypothermosol, have led to studies showing significant improvement in preservation efficacy.

Anti-apoptotic agents may be selected from those that possess recognized antioxidant activities and hence implied anti-apoptotic activity. For example, reduced glutathione is a component of both formulations as a multifaceted molecule that is also known to fulfill a natural role in the regulation of apoptosis, bongkrekic acid (BA) has been shown to be a potent inhibitor of mitochondrial permeability transition (PT) pores that form during apoptosis. In addition, BA can inhibit cytochrome c release that is influenced by Bax, a pro-apoptotic protein 85. BA, a stable inhibitor of PT, has been shown to increase cell viabilities and protein production levels following virus infection. With respect to the inhibition of caspases, a variety of compounds have been shown to be effective for mammalian cells in culture. Other exemplary compounds include, P35, which confers irreversible inhibition to a large number of caspases, and Z-VAD.fmk (or its latest broad-spectrum counterpart, Q-VD-OPH), which has the ability to inhibit both the intrinsic and extrinsic pathways.

Trophic Factors: Many cell signaling pathways retain activity at very low temperatures and can be affected by trophic factor administration. Trophic factor deprivation disrupts many aspects of cell function and is well known to induce apoptosis and cell death in a wide variety of cultured cells. Trophic factor supplementation (TFS) leads to a markedly improved outcome in kidney storage an influence cold ischemic injury by interaction with the tissue during cold storage and not merely by being present during re-warming and reperfusion. Exemplary tropic factors, which may be employed include, for example, Insulin-like growth factor-1 (IGF-1) Epidermal growth factor (EGF), Bovine neutrophil peptide-1 (BNP-1), also referred to as bactenecin 98, Substance P (SP), which has mitogenic effects for a variety of cell types and stimulates DNA synthesis in ocular cell lines, EGF, a polypeptide growth factor (its effects may be additive or synergistic with other growth factors and cytokines), and insulin-like polypeptide growth factors (IGFs), such as IGF-1.

In embodiments, the PFCs may possess one or more of the following qualities: (1) the ability to dissolve large quantities of many gases, (2) can transport these gases to diffuse across distances, (3) are non-toxic, (4) biologically inert, (5) biostatic liquids at room temperature. In embodiments, PFCs with densities of about 1.5-2.0 g/mL and high solubilities for oxygen and carbon dioxide may be selected.

In embodiments, for a variety of reasons, such as preservation and transport, the donor tissue may be cooled to a sufficient temperature to attenuate metabolism, such as a temperature of from about 15° C. to about −20° C., such as from about 10° C. to about −10° C., or from about 10° C. to about 0° C. In embodiments, the cooling rate may be from about 0.5° C./min. to about 5° C./min. In embodiments, freezing and/or cooling the donor tissue may occur at a cooling rate of from about 1° C./min. to about 20° C./min., such as from about 6° C./min. to about 15° C./min.

In embodiments, the rate of cooling the donor tissue coupled with a rapid warming rate (such as the above rates for cooling multiplied by a factor of at least 1.5, such as a factor in the range from 1.5 to 10, such as a factor of 2, or 3, or 4, or 5) during warming of the donor tissue. Warming of the donor tissue may be achieved by, in embodiments, direct immersion in a warm medium, such as an osmotically-buffered medium.

Examples of other additives that may be used are listed in Table 3, although many other additives can be used.

Exemplary solutions to which at least one complement inhibitor may be added for a clinical organ preservation program are summarized in Table 1.

TABLE 1
Solution Design Strategy for Clinical Organ Preservation Program

| | Temperature | Base | |
|---|---|---|---|
| Application Phase | Range | Solution Type | Additives |
| Organ Preparation | 10-37° C. | Extracellular | Purging cocktail |
| in situ/ex vivo purge | | | |
| Organ Maintenance | 0-4° C. | Intracellular | Protection/ |
| cold flush | | (High K) | Maintenance |
| | | | cocktail |
| Organ Maintenance | 5-15° C. | Intracellular | Protection/ |
| cold machine | | (High K | Maintenance |
| perfusion or | | or Na) | cocktail |
| cardiopulmonary | | | |
| bypass | | | |
| Organ Rescue | 30-37° C. | Extracellular | Rescue cocktail |
| warm machine | | | |
| perfusion | | | |
| Organ | 30-37° C. | Extracellular | Plasma-like |
| Pre-reimplantation | | | |
| ex vivo rinse | | | |

TABLE 2
Formulation of unified solution system Intracellular Base (High Potassium)

| | | | | | | | |
|---|---|---|---|---|---|---|---|
| Na⁺ | 62.5 mM | Cl⁻ | 30.1 mM | HEPES | 35 mM | Dextran - 40 | 6% |
| K⁺ | 70 mM | H₂PO₄ | 2.5 mM | Lactobionate | 30 mM | Glucose | 5 mM |
| Ca²⁺ | 0.05 mM | HCO₃⁻ | 5 mM | Gluconate | 70 mM | Adenosine | 2 mM |
| Mg²⁺ | 15 mM | | | Sucrose | 15 mM | reduced Glutathione | 3 mM |
| | | | | Mannitol | 25 mM | | |

TABLE 3
Exemplary Biochemical and Pharmacological Additives for
Preservation Media

| Classification | Examples |
|---|---|
| Anti-platelet aggregation/ | Prostacyclin, Prostaglandin E-1 (PGE1), |
| vasoactive agents | Mg²⁺ |
| Calmodulin inhibitors | Chlorpromazine (CPZ), trifluoperazine |
| Calcium Channel Blockers | Nicardipine, nifedipine, verapamil, CPZ |
| Protease and phospholipase | CPZ, verapamil, calpain antagonists |
| inhibitors | |
| Anti-oxidants/free radical | Glutathione, catalase, super oxide dismutase |
| scavengers | (SOD), allopurinol, dimethylthiourea, |
| | vitamin-E (or Trolox), magnesium ascorbyl |
| | phosphate, Lazaroids |
| Anti-apoptotic agents | cycloheximide |
| Iron chelators | Desferroxamine |
| Membrane Stabilizers | CPZ, Dexamethosone, trehalose |
| “Cytoprotective” agents | PGE1, glycine |
| Metabolic Substrates: | |
| Sugars | glucose, fructose, ribose |
| Nucleotide precursors (HEP | Adenine, Adenosine, Fructose diphosophate, |
| enhancers) | Glyceraldehyde-3-phosphate |
| Oxygen-carriers | Perfluorocarbons, PEG-hemoglobin |
| Trophic Factors | Growth factors, nucleic acid derivatives, |
| | ribonucleotides, glycosaminoglycans |
| Cryoprotective Additives | Dimethylsulfoxide (DMSO), glycerol, |
| (CPA) | propanediol, ethylene glycol, butanediol, |
| | polyvinylpyrrolidone (PVP), hydroxyethyl |
| | starch (HES), polyethylene glycol (PEG) |

A substantial number of improvements in the combinations of components and their respective concentrations have been incorporated in the design of the compositions and systems of the methods of the present disclosure over that of the known art, including Hypothermosol™.

Exemplary aqueous formulations of both intracellular and extracellular base solutions into which at least one complement inhibitor (and other additives) may be added are illustrated below. The formulations can contain substantially about the amounts listed.

### Exemplary Intracellular Base Solutions

Ionic

40-80 mM Na⁺;

50-90 mM K⁺;

0.01-0.1 mM Ca⁺⁺;

5-25 mM Mg⁺⁺;

20-40 mM Cl⁻;

pH Buffers

1-5 mM H₂PO₄;

3-7 mM HCO₃;

25-50 mM HEPES;

Impermeants

25-50 mM Lactobionate;

10 mM-1M Sucrose;

15-30 Mannitol;

1-10 Glucose;

50-100 Gluconate;

Colloids

6% Dextran 40;

Pharmacologics

0.1-2 mM Adenosine; and

1-5 mM Glutathione.

### High Potassium Exemplary Intracellular Base Solution

Ionic

62.5 mM Na⁺;

70.0 mM K⁺;

0.05 mM Ca⁺⁺;

15.0 mM Mg⁺⁺;

30.1 mM Cl⁻;

pH Buffers

2.5 mM H₂PO₄;

5.0 mM HCO₃;

35.0 mM HEPES;

Impermeants

30.0 mM Lactobionate;

15.0 mM Sucrose;

25.0 mM Mannitol;

5.0 mM Glucose;

70.0 mM Gluconate;

Colloids

6% Dextran 40;

Pharmacologics

2.0 mM Adenosine; and

3.0 mM Glutathione.

This exemplary Intracellular Base Solution has an osmolality (mOsm/Kg) of 350, a pH of about 7.6, and a [K⁺][Cl⁻] of about 2100.

### Low Potassium Exemplary Intracellular Base Solutions

Ionic

100-150 mM Na⁺;

15-40 mM K⁺;

0.01-0.1 mM Ca⁺⁺;

5-25 mM Mg⁺⁺;

20-40 mM Cl⁻;

pH Buffers

1-5 mM H₂PO₄;

3-7 mM HCO₃;

25-50 mM HEPES;

Impermeants

25-50 mM Lactobionate;

10 mM-1M Sucrose;

15-30 mM Mannitol;

1-10 mM Glucose;

50-100 mM Gluconate;

Colloids

6% Dextran 40;

Pharmacologics

0.1-2 mM Adenosine; and

1-5 mM Glutathione.

Exemplary Low Potassium Intracellular Solutions

Ionic

125 mM Na⁺;

25.0 mM K⁺;

0.05 mM Ca⁺⁺;

15.0 mM Mg⁺⁺;

30.1 mM Cl⁻;

pH Buffers

2.5 mM H₂PO₄;

5.0 mM HCO₃;

35.0 mM HEPES;

Impermeants

30.0 mM Lactobionate;

15.0 mM Sucrose;

25.0 mM Mannitol;

5.0 mM Glucose;

70.0 mM Gluconate;

Colloids

6% Dextran 40;

Pharmacologics

2.0 mM Adenosine; and

3.0 mM Glutathione.

### Exemplary Extracellular Base Solutions

Ionic

120-160 mM Na⁺;

3-9 mM K⁺;

1-3 mM Ca⁺⁺;

1-10 mM Mg⁺⁺;

100-150 mM Cl⁻;

1-10 mM (SO₄)²⁻;

pH Buffers

1-3 mM H₂PO₄;

20-30 mM HCO₃;

5-15 mM HEPES;

Impermeants

5-10 mM Glucose;

Colloids

6% Dextran 40;

Pharmacologics

0.1-2 mM Adenosine; and

1-5 mM Glutathione.

### Exemplary Extracellular Base Solution

Ionic

141.2 mM Na⁺;

6.0 mM K⁺;

1.5 mM Ca⁺⁺;

5.0 mM Mg⁺⁺;

122.0 mM Cl⁻;

1.0 mM SO₄;

pH Buffers

1.2 mM H₂PO₄;

25.0 mM HCO₃;

25.0 mM HEPES;

Impermeants

5.0 mM Glucose;

Colloids

6% Dextran 40;

Pharmacologics

1.0 mM Adenosine; and

3.0 mM Glutathione.

This exemplary extracellular base solution has an osmolality (mOsmlKg) of 315, a pH of about 7.5, and a [K⁺][Cl⁻] of about 732.

As outlined above, the strategic designs of solutions used for organ preservation have differed depending upon their ultimate use, either as perfusates for continuous, or intermittent, perfusion of the organ. As a unique approach, the methods of the present disclosure, which include the addition of at least one complement inhibitor to the various solutions described herein, have been developed with a view to improving techniques that may be used for machine perfusion preservation. An attempt has been made to combine the main characteristics of effective solutions, such as hypothermic solutions, in the formulation of the base solution (which includes at least one complement inhibitor), and wherever possible, components, including, inter alia, complement inhibitors, that might fulfill multiple roles have been selected. For example, an extracellular base solution comprising at least one complement inhibitor may be combined with various different additives to form purging solutions, organ rescue solutions, pre-implantation rinses and the like. This strategy maximizes the intrinsic qualities of the machine perfusion preservation process.

In embodiments, the solutions to be perfused may be held in a reservoir of the perfusion apparatus and may be brought to a predetermined temperature by a first thermoelectric unit in heat transfer communication with the reservoir. A temperature sensor may relay the temperature within the reservoir to the microprocessor, which adjusts some in turn the thermoelectric unit to maintain a desired temperature within the reservoir and/or display the temperature on a control and display area for manual adjustment. Alternatively, or in addition, particularly where the organ perfusion device is going to be transported, the solution within the reservoir may be cooled utilizing a cryogenic fluid heat exchanger apparatus such as that disclosed in U.S. Pat. No. 6,014,864, the disclosure of which is hereby incorporated by reference herein in its entirety.

In embodiments, optimum control of the intracellular and extracellular environment of cells during hypothermia may depend upon optimizing the interaction of a variety of factors that include temperature, oxygen tension, acidity, osmotic pressure and chemical composition (including complement inhibitor concentration) of the perfusion solution.

In embodiments, the successive phases of the transplantation procedure involving organ procurement, storage, transportation, reimplantation and reperfusion may impose different requirements for optimum preservation at the different stages, and thus require perfusion solutions comprising different compositions. Therefore, any single formulation of preservation solution is unlikely to provide optimum protection during all the processing stages of a transplantation procedure, or the interventional stages of complex surgeries. In embodiments, the changes in the solution may be made during perfusion in any manner, such as by way of a stepwise change, or gradient change in which a respective component's concentration is gradually increased to the desired concentration, or decreased to the desired concentration, or decreased to the point of elimination from the solution being perfused, if necessary.

In embodiments, an organ chamber may be provided which supports a cassette. The cassette may be configured to hold an organ to be perfused. Otherwise the organ chamber may support a plurality of cassettes, which may be disposed one adjacent the other. The cassette may be formed of a material that is light but durable so that the cassette is highly portable. The material may also be transparent to allow visual inspection of the organ.

In embodiments, the cassette may include side walls, a bottom wall and an organ supporting surface. The organ supporting surface may be formed of a porous, perforated or mesh material to allow fluids to pass through. The cassette may also include a top and may be provided with one or more openings for tubing. The openings may include seals, e.g., septum seals or o-ring seals and optionally be provided with plugs to prevent contamination of the organ and maintain a sterile environment. In embodiments, the cassette may be provided with a closeable and/or vent. Additionally, the cassette may be provided with tubing for connection to the organ and/or to remove perfusion solution and/or medical fluid from an organ bath, and one or more connection devices for connecting the tubing to, for example, tubing of an organ storage, transporter, perfusion and/or diagnostic apparatus.

In embodiments, the apparatus may comprise a vent that may include a filter device, and provide for control and/or equalization of pressure within the cassette without contamination of the contents of the cassette. For example, organs are frequently transported by aircraft, in which pressure changes are the norm. Even ground transportation can involve pressure changes as motor vehicles pass through tunnels, over mountains, etc. In addition, one or more lids of the cassette may create an airtight seal with the cassette. This air tight seal can create a pressure difference between the inside and outside of the cassette. It may be desirable to provide for pressure equalization of the cassette under such circumstances. However, free flow of air to achieve pressure equalization might introduce contaminants into the cassette. Thus, a vent including a filter may be provided to allow the air flow without permitting introduction of contaminants into the cassette.

The filter may facilitate clean air passing in both directions, while restricting dirt, dust, liquids and other contaminants from passing. The pore size of the filter can be selected to prevent bacteria from passing.

A pressure control valve (not shown) may optionally be associated with vent as well. Such a valve may be configured and controlled to restrict the rate at which external pressure changes are transmitted to the inside of the cassette, or even to prevent pressure increases and/or decreases, as desired.

The cassette, and/or the organ supporting surface, openings, tubings and/or connection device, may be specifically tailored to the type of organ and/or size of organ to be perfused. Flanges of side support walls may be used to support the cassette disposed in an organ storage, transporter, perfusion and/or diagnostic apparatus. The cassette may further include a handle that allows the cassette to be easily handled. Each cassette may also be provided with its own mechanism, e.g., stepping motor/cam valve for fine tuning the pressure of solution perfused into the organ, as discussed in more detail below. Alternatively, or in addition, pressure may, in embodiments, be controlled by way of a microprocessor, which may receive pressure sensor data from a pressure sensor. Likewise, flow sensors may be controlled in a similar manner.

A biopsy and/or venting port may be included in inner lid, or in both an inner lid and an outer lid. A port may provide access to the organ to allow for additional diagnosis of the organ with minimal disturbance of the organ. Cassette may also have an overflow trough. The overflow trough may provide a region to check to determine if the inner seal is leaking. Perfusate may be poured into and out of cassette and may be drained from the cassette through a stopcock or removable plug.

The cassette may be constructed of an optically transparent material to allow for viewing of the interior of cassette and monitoring of the organ and to allow for video images or photographs to be taken of the organ. A perfusion apparatus or cassette may be wired and fitted with a video camera or a photographic camera, digital or otherwise, to record the progress and status of the organ. Captured images may be made available over a computer network such as a local area network or the Internet to provide for additional data analysis and remote monitoring. The cassette may also be provided with a tag that would signal, e.g., through a bar code, magnetism, radio frequency, or other means, the location of the cassette, that the cassette is in an the apparatus, and/or the identity of the organ to perfusion, storage, diagnostic and/or transport apparatus. Cassette may be sterile packaged and/or may be packaged or sold as a single-use disposable cassette, such as in a peel-open pouch. A single-use package containing the cassette may also include a tubeset and/or tube frame.

In embodiments, the cassette may be configured such that it may be removed from an organ perfusion apparatus and transported to another organ perfusion and/or diagnostic apparatus in a portable transporter apparatus as described herein or, for example, a conventional cooler or a portable container such as that disclosed in U.S. Pat. No. 6,209,343, or U.S. Pat. No. 5,586,438 to Fahy, the disclosures of which are hereby incorporated by reference herein in their entirety.

In various exemplary embodiments, when transported, the organ may be disposed on the organ supporting surface and the cassette may be enclosed in a sterile bag. When the organ is perfused with a solution, which may comprise at least one complement inhibitor, effluent collects in the bag to form an organ bath. Alternatively, cassette 65 may be formed with a fluid tight lower portion in which effluent solution or medical fluid may collect, or effluent solution or medical fluid may collect in another compartment of an organ storage, transporter, perfusion and/or diagnostic apparatus, to form an organ bath. The bag may be removed prior to inserting the cassette into an organ storage, transporter, perfusion and/or diagnostic apparatus. Further, where a plurality of organs are to be perfused, multiple organ compartments may be provided.

The transporter may have a stable base to facilitate maintaining an upright position and handles for carrying the transporter. The transporter may also be fitted with a shoulder strap and/or wheels to assist in carrying transporter. A control panel may be provided. The control panel may display characteristics, such as, but not limited to, infusion pressure, attachment of the tube frame, power on/off, error or fault conditions, flow rate, flow resistance, infusion temperature, bath temperature, pumping time, battery charge, temperature profile (maximums and minimums), cover open or closed, history log or graph, and additional status details and messages, some or all of which may be further transmittable to a remote location for data storage and/or analysis. Flow and pressure sensors or transducers in transporter may be provided to monitor various organ characteristics including pump pressure and vascular resistance of an organ, which can be stored in computer memory to allow for analysis of, for example, vascular resistance history, as well as to detect faults in the apparatus, such as elevated pressure.

The transporter may include latches that require positive user action to open, thus avoiding the possibility that transporter inadvertently opens during transport. Latches may hold the top in place on transporter. The top or a portion thereof may be constructed with an optically transparent material to provide for viewing of the cassette and organ perfusion status. The transporter may be configured with a cover open detector that monitors and displays whether the cover is open or closed. The transporter may be configured with an insulating exterior of various thicknesses to allow the user to configure or select an appropriate transporter for varying extents and distances of transport. In embodiments, a compartment may be provided to hold patient and organ data such as charts, testing supplies, additional batteries, hand-held computing devices and/or configured with means for displaying a UNOS label and/or identification and return shipping information.

The transporter may be fitted with, a conformed cassette and include pump. The cassette may be placed into or taken out of transporter without disconnecting a tubeset from cassette, thus maintaining sterility of the organ. In embodiments, sensors in the transporter can detect the presence of the cassette in the transporter, and, depending on the sensors, can read the organ identity from a barcode or radio frequency or other “smart” tag that may be attached, or integral, to the cassette. This can allow for automated identification and tracking of the organ in the cassette, and helps to monitor and control the chain of custody. A global positioning system receiver may be added to the transporter and/or cassette to facilitate tracking of the organ. The transporter may be interfaceable to a computer network by hardwire connection to a local area network or by wireless communication, for example, while in transit. This interface may allow data such as perfusion parameters, vascular resistance, and organ identification, and the transporter and cassette location, to be tracked and displayed in real-time or captured for future analysis.

The transporter may contain a filter to remove sediment and other particulate matter from the perfusate to prevent clogging of the apparatus or the organ. The transporter may also contains batteries, which may be located at the bottom of the transporter or beneath pump or at any other location that provides easy access to change batteries. The transporter may also provide an additional storage space, for example, at the bottom of the transporter, for power cords, batteries and other accessories. Transporter may also include a power port for a DC hookup, e.g., to a vehicle such as an automobile or airplane, and/or for an AC hookup.

The transporter may have an outer enclosure, which may, for example, be constructed of metal, plastic or synthetic resin that is sufficiently strong to withstand penetration and impact. The transporter may contain insulation, such as a thermal insulation made of, for example, glass wool or expanded polystyrene. The insulation may be of various thicknesses. The transporter may be cooled by coolant, which may be, e.g., an ice and water bath or a cryogenic material. In embodiments using cryogenic materials, the design should be such that organ freezing is prevented. The transporter may be configured to hold various amounts of coolant. An ice and water bath is preferable because it is inexpensive and generally cannot get cold enough to freeze the organ. The level of coolant may, for example, be viewed through a transparent region of the transporter or be automatically detected and monitored by a sensor. The coolant may be replaced without stopping perfusion or removing cassette from transporter. The coolant may be maintained in a fluid-tight compartment of the transporter. The coolant may provide a failsafe cooling mechanism where the transporter automatically reverts to cold storage in the case of power loss or electrical or computer malfunction. The transporter may also be configured with a heater to raise the temperature of the perfusate.

The transporter may be used to perfuse various organs such as a kidney, heart, liver, small intestine and lung. The transporter and cassette may accommodate various amounts to perfusate, which may be selected as desired.

The cassette and transporter may be constructed to fit or mate such that efficient heat transfer is enabled. The transporter may rely on conduction to move heat from the cassette to the coolant contained in a predetermined compartment. This movement of heat allows the transporter to maintain a desired temperature of the perfusion solution. The geometric elements of the cassette and transporter may be configured such that when the cassette is placed within transporter, the contact area between the cassette and the transporter is as large as possible and they are secured for transport.

The pump, which may be a peristaltic pump, or any type of controllable pump, may be used to move fluid throughout the infusion circuit of, for example, the organ perfusion apparatus, organ cassette, and/or the organ transporter, and into the organ.

It should be appreciated that the organ may be any type of organ, a kidney, liver, or pancreas, for example, and the organ may be from any species, such as a human or other animal.

In a flow path for perfusate (infusion circuit), immediately preceding, or within, the organ, may lie a pressure sensor, which can sense the pressure of fluid flow at the position before the fluid enters, or dispersed in, the organ. As fluid is moved throughout the infusion circuit, the organ provides resistance. The pressure sensor may detect the pressure that the organ creates by this resistance as the fluid moves through it. At a position after organ, there is little pressure, as the fluid typically flows out of the organ freely and into an organ bath.

The combination of flow and pressure in a vessel generally describe a therapeutic window for organ perfusion. If the flow or pressure is too high, the organ may be damaged; if the flow or pressure is too low, then the therapeutic benefits of perfusion are not realized at all, or as fully as desired. The therapeutic window is empirical, and may be established within an organ perfusion apparatus by a user or manufacturer. For example, the user may set a target value that each machine may attempt to maintain while maintaining a parameter according to a preset therapeutic value. For example, each machine may attempt to maintain a target pressure while maintaining flow above a therapeutic minimum. The organ perfusion apparatus is directed to maintaining pressure and flow within the therapeutic window.

For comparative reference, generally organs, such as kidneys, for example, are considered two-terminal devices. In a kidney, flow enters the renal artery and exits the renal vein. Flow through the kidney can be increased or decreased by increasing or decreasing the fluid pressure going into the renal artery. During perfusion, higher fluid pressure at the entrance to the renal artery delivers higher flow into the renal artery. Because the relationship between pressure and flow changes during perfusion as blood vessels tighten and loosen, an automatic controller within a kidney perfusion apparatus continually measures and adjusts pressure and flow up or down to stay within the therapeutic window. This mode of controller operation can be considered the Linear mode.

During relatively low flow conditions, such as may be encountered at the start of perfusion when the blood vessels are constricted in a liver, the flow into the portal vein and hepatic artery of a liver may be controlled independently using the Linear mode (as described for the kidney above). The pressure and flow of each vessel may be raised and lowered independently to maintain operation within appropriate therapeutic windows.

As the flow into the hepatic artery increases, a threshold is reached (which varies from liver to liver), at which further increase in hepatic artery flow may result in reduction of portal vein flow. This is flow competition. Flow competition may increase to a degree that it drives the parameter for the fluid flowing in the portal vein below the therapeutic minimum even though the portal vein pressure has reached the therapeutic maximum. At this point, further control of the portal vein within the therapeutic window cannot be maintained by adjusting the portal vein machine alone. A perfusion apparatus including the systems and methods according to this disclosure may detect such conditions and the deformentation may be made that a controller should direct the perfusion apparatus to a Cooperative mode of operation, with a goal of coordinated maintenance of both hepatic artery and portal vein pressure and flow within appropriate therapeutic windows.

Pressure and flow of a therapeutic window may be established with user settings, in apparatus pre-sets, as stored parameters, and/or combinations thereof. A user or computer sets a desired systolic pressure and/or flow, which is the pressure and flow of the fluid supply before entering the organ at a pressure sensor. A peristaltic pump, for example, or any other type of controllable pump, may be used in embodiments of the present disclosure.

All patents, patent applications, scientific articles and other sources and references cited herein are explicitly incorporated by reference herein for the full extent of their teachings as if set forth in their entirety explicitly in this application.

It should be understood that the foregoing disclosure emphasizes certain specific embodiments of the invention and that modifications or alternatives equivalent thereto are within the spirit and scope of the invention.

What is claimed is:

1. A method of preserving an organ or tissue, comprising the steps of:
(a) providing a base perfusion solution;
(b) mixing a first portion of the base perfusion solution with at least one complement inhibitor to produce a first mixed solution; and
(c) perfusing an organ or tissue during at least one stage of procurement, preservation or transplantation of that organ or tissue with the first mixed solution.

2. The method of claim 1, wherein the organ or tissue is perfused with the first mixed solution while the organ or tissue is being warmed from a temperature below about 0° C. to a temperature in the range from about 10° C. to about 37° C.

3. The method of claim 1, wherein the organ or tissue is perfused with the first mixed solution while the organ or tissue is being warmed from a temperature below about 10° C. to a temperature in the range from about 25° C. to about 37° C.

4. The method of claim 1, wherein the organ or tissue is perfused with the first mixed solution without previously cooling the organ to a temperature below about 25° C.

5. The method of claim 1, wherein the organ or tissue is perfused with the first mixed solution while the organ or tissue is maintained at a temperature range from about 10° C. to about 37° C.

6. The method of claim 1, wherein the organ or tissue of step (c) is not exposed to a non-native complement inhibitor before perfusion, with the first mixed solution.

7. The method of claim 1, wherein the organ or tissue of step (c) is exposed to a non-native complement inhibitor before perfusion with the first mixed solution.

8. The method of claim 1, wherein said base perfusion solution comprises an intracellular base solution.

9. The method of claim 1, wherein said base perfusion solution comprises an extracellular base solution.

10. The method of claim 1, wherein perfusing an organ or tissue during at least one stage of procurement, preservation or transplantation of that organ or tissue with the first mixed solution occurs after the organ or tissue has been cooled to a temperature less than 10° C.

11. The method of claim 1, wherein said base solution is an intracellular base solution that comprises a cytoprotective additive that forms a maintenance solution from said intracellular base solution.

12. The method of claim 1, wherein step (c) comprises restoring viability of said organ or tissue by near normothermic perfusion; and
wherein said base solution is an extracellular base solution comprising a rescue additive.

13. The method of claim 1, wherein step (c) comprises flushing unwanted molecules away from an organ or tissue;
and wherein said base perfusion solution is an extracellular base solution comprising a rinse additive.

14. The method of claim 13, wherein said unwanted molecules are preservation solution molecules, and step (c) is followed by transplantation of the organ or tissue.

15. The method of claim 13, wherein said unwanted molecules are erythrocytes and other blood components, and step (c) is followed by a preservation process.

16. The method of claim 1, wherein step (c) comprises cryopreservation of said organ or tissue by sub-zero cooling;
and wherein said base perfusion solution is an intracellular base solution, and said first additive is a cryoprotective additive.

17. The method of claim 1, wherein said base perfusion solution comprises:
Na⁺;
K⁺;
Mg⁺⁺;
Cl⁻;
H₂PO₄⁻;
HCO₃⁻;
HEPES;
Lactobionate;
Sucrose;
Mannitol;
Glucose;
Gluconate;
Dextran 40;
Adenosine; and optionally
Glutathione.

18. The method of claim 1, wherein the base perfusion solution does not contain Glutathione.

19. The method of claim 1, wherein the base perfusion solution is an oxygen carrying solution.

20. The method of claim 1, wherein the base perfusion solution is a non-oxygen carrying solution.

21. The method of claim 1, wherein the first mixed solution contains an effective amount of the at least one complement inhibitor to substantially prevent detrimental responses that can injure the organ or tissue, or recipient, including precipitating or enhancing an immunological reaction from the recipient.

22. The method of claim 1, wherein the first mixed solution is perfused so as to deliver a homogeneous distribution of the at least one complement inhibitor throughout the organ or tissue.

23. The method of claim 1, wherein the base perfusion solution comprises perfluorochemicals.

24. The method of claim 23, wherein the perfluorochemicals represent from about 10% to about 90% of the total weight of the base perfusion solution.

25. The method of claim 1, wherein the at least one complement inhibitor represents from about 0.00001% to about 0.1% of the total weight of the base perfusion solution.

26. The method of claim 1, wherein the base perfusion solution comprises cytoprotective additives.

27. The method of claim 26, wherein the cytoprotective additives are one or more additive selected from the group consisting of antioxidants, anti-apoptotic agents and trophic factors.

28. The method of claim 1, wherein the base perfusion solution is a hypothermic blood substitute, the hypothermic blood substitute comprising: cytoprotective agents, and perfluorochemicals.

29. The method of claim 1, further comprising a step of increasing the ATP levels in the donor tissues during perfusion.

30. The method of claim 1, further comprising introducing cytoprotective agents during step (c) for preventing cold-induced cell death of the donor tissue.

31. The method of claim 1, further comprising introducing cytoprotective agents during step (c) for preventing cells of a donor organ from entering destructive pathways.

32. The method of claim 1, further comprising introducing cytoprotective agents during step (c) for inhibiting mitochondrial dysfunction in cells of a donor organ.

33. The method of claim 1, further comprising preventing anaerobic glycolysis in the organ or tissue.

34. The method of claim 33, wherein preventing anaerobic glycolysis in the organ or tissue comprises introducing perfluorochemicals into the perfusion solution.

35. The method of claim 1, further comprising preventing oxygen deprivation/depletion in the organ or tissue.

36. The method of claim 35, wherein preventing oxygen deprivation/depletion in the organ or tissue comprises introducing perfluorochemicals into the perfusion solution.

37. The method of claim 1, further comprising disconnecting the perfusion apparatus from the donor tissue.

38. The method of claim 37, further comprising satisfying the O₂ demand of a donor tissue throughout a preservation interval/process occurring from the time the perfusion apparatus is connected to the donor tissue to the time perfusion apparatus is disconnected from the donor tissue.

39. The method of claim 1, further comprising replenishing O₂ content in the first mixed solution during step (c).

40. The method of claim 1, further comprising increasing O₂ content in the first mixed solution during step (c).

41. The method of claim 1, further comprising monitoring complement inhibitor coverage of the tissue or organ during step (c).

42. The method of claim 1, further comprising assessing and monitoring the concentration of interstitial fluid components.

43. The method of claim 42, wherein the interstitial fluid components are selected from the group consisting of glucose, lactate, pyruvate, glycerol, ATP, O₂ and CO₂.

44. The method of claim 1, wherein said organ or tissue is from a mammal.

45. The method of claim 1, wherein said organ or tissue is from a non-heart beating donor.

46. The method of claim 1, wherein said organ or tissue is from a heart beating.

47. The method of claim 1, wherein the organ is selected from the group consisting of a kidney, a liver, a heart, and a pancreas.

48. The method of claim 1, wherein the base perfusion solution does not contain any complement inhibitors.
